(19) United States
(12) Patent Application Publication
(10) Pub. No.: US 2026/0008796 A1
(43) Pub. Date: Jan. 8, 2026

(54) INDANONE DERIVATIVES AS NUCLEOBASE BUILDINGS BLOCK FOR ON-DNA ALDEHYDE CAPTURE

(71) Applicant: UNIVERSITY OF GUELPH, Guelph (CA)

(72) Inventors: Richard MANDERVILLE, Guelph (CA); Ryan JOHNSON, Guelph (CA)

(21) Appl. No.: 18/881,808

(22) PCT Filed: Jul. 7, 2023
(86) PCT No.: PCT/CA2023/050921

§ 371 Date: Jan. 7, 2025

Related U.S. Application Data
(60) Provisional application No. 63/359,878, filed on Jul. 10, 2022.

(51) Int. Cl. C07F 9/22; C07F 9/24; C12Q 1/6816

(52) U.S. Cl. CPC C07F 9/222; C07F 9/2429; C12Q 1/6816

(57) ABSTRACT

The present disclosure relates to compounds of Formula (I) which are incorporated into DNA for on-DNA aldehyde capture and use as fluorescent molecular rotors.

### CROSS-REFERENCE TO RELATED APPLICATION

This application claims the benefit of priority to U.S. Provisional Application No. 63/359,878, filed Jul. 10, 2022, the contents of which is incorporated herein by reference in its entirety.

### FIELD

The present disclosure relates to compounds of Formula (I) which are incorporated into DNA for on-DNA aldehyde capture and use as fluorescent molecular rotors.

### INTRODUCTION

Fluorescent probes are critical tools for detecting and sensing the structure, dynamics, and binding interactions of nucleic acids. For these applications, fluorescent molecular rotors (FMRs) are highly valuable because they possess segmental mobility, with enhanced dye rigidity leading to light-up fluorescence.¹ Free cationic FMRs, such as thioflavin-T (ThT),² thiazole orange (TO)³ and hemicyanines,^{4,5} are extensively utilized for DNA sensing applications. They are commercially available, or readily synthesized from affordable building blocks, making them relatively simple for end-users to employ. However, despite their ease of use and remarkable performance, they cannot provide site-specific information and can suffer from false fluorescent readouts because they are not covalently attached to the biopolymer.

Nucleic acid chemists have also developed numerous covalent FMR probes for incorporation site-specifically into nucleic acids.⁶ These molecular reporters can provide direct fluorescent readouts for hydrogen-bonding,⁷ metal binding,⁸ hybridization,⁹⁻¹¹ topology switching,¹² changes in pH,¹³ microviscosity and for monitoring binding interactions that are resistant to false readouts.^{14,15} Despite these numerous advantages, they are seldom employed in DNA sensing applications. Issues that limit their wide-spread utility include that they exist in several varieties, making it difficult to know which type to choose for a given application. For example, canonical derivatives which maintain Watson-Crick (W-C) base-pairing may be extended or isomorphic. Extended derivatives contain an FMR covalently tethered, via a flexible non-fluorescent linker, to a natural nucleobase,^{16,17} while isomorphic probes are nucleobase analogues.^{7,13} Alternatively, non-canonical derivatives may be employed that lack base-pairing properties and serve as nucleobase surrogates.^{15,18} In each case, the FMR must either be converted into a phosphoramidite for solid-phase synthesis, or a 5′-triphosphate for enzymatic incorporation site-specifically into the oligonucleotide of interest. Such building blocks are not commercially available, and many researchers involved in nucleic acid sensing lack the expertise to synthesize such specialized analogues.⁶

### SUMMARY

The present disclosure is directed to compounds of the Formula (I) having the structure

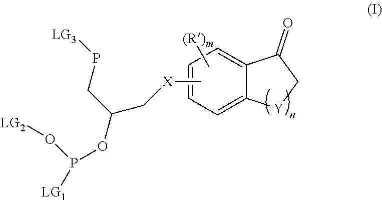

wherein
X is O or NR₁, wherein R₁ is H or (C₁-C₆)-alkyl;
Y is —C(R)(R)—, O, S, or NR₂, wherein R₂ is H or (C₁-C₆)-alkyl;
each R is independently or simultaneously H, halo, (C₁-C₆)-alkyl or (C₁-C₆)-alkoxy;
R′ is a substituent on the benzene ring and is halo, (C₁-C₆)-alkyl or (C₁-C₆)-alkoxy, and m is 0, 1, 2 or 3;
LG₁, LG₂ and LG₃ are each suitable leaving groups;
n is the integer 1, 2 or 3; and
any stereoisomers thereof.

In another embodiment of the disclosure, the compounds of the Formula (I) are used as nucleobase building blocks to prepare oligomers of DNA of the Formula (II) incorporating the compounds of Formula (I) and can be condensed with reactive aldehyde groups:

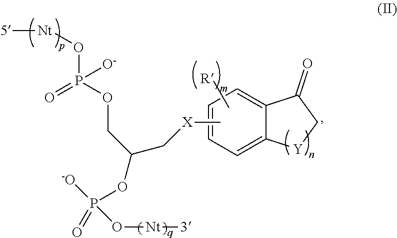

wherein,
X is O or NR₁, wherein R₁ is H or (C₁-C₆)-alkyl;
Y is —C(R)(R)—, O, S, or NR₂, wherein R₂ is H or (C₁-C₆)-alkyl;
each R is independently or simultaneously H, halo, (C₁-C₆)-alkyl or (C₁-C₆)-alkoxy;
R′ is a substituent on the benzene ring and is halo, (C₁-C₆)-alkyl or (C₁-C₆)-alkoxy,
and m is 0, 1, 2 or 3;
n is the integer 1, 2 or 3;
wherein Nt is a nucleotide;
m and p are independently an integer between 1 and 1000; and
any stereoisomers thereof.

In another embodiment, there is included a chalcone-derivatized oligonucleotide of the Formula (III) having the structure

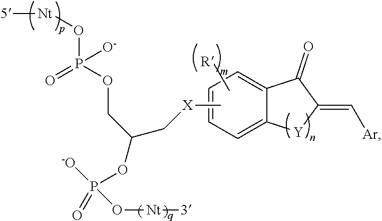

wherein,
Ar is an aromatic group or moiety, forming a chalcone moiety or derivative; and
Nt, p, q, R′, m, n and Y are as defined above.

In one embodiment, the moiety forming the chalcone moiety is

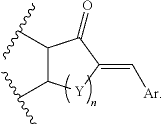

In another embodiment, the present disclosure includes a method for the detection of nucleic acid hybridization to create duplex nucleic acids (DNA and/or RNA) comprising,
a) contacting an oligonucleotide having the structure of Formula (II) with a reactive aldehyde compound to obtain a chalcone-derivatized oligonucleotide;
b) contacting the chalcone-derivatized oligonucleotide with a complementary nucleic acid (DNA and/or RNA) through hybridization to afford a duplex nucleic acid structure; and
c) measuring an increase of fluorescence intensity of the duplex nucleic acid structure.

In another embodiment, there is included a method of detecting a nucleic acid, comprising
a) contacting a sample with a chalcone derivatized oligonucleotide having the structure of Formula (III);
b) detecting a fluorescence signal at a wavelength specific for the chalcone moiety;
c) comparing the fluorescence signal of (b) with the fluorescence intensity of a control sample;
   wherein detection of a signal in the sample having a fluorescence intensity greater than the control sample indicates the sample contains the nucleic acid.

In another embodiment, derivatized oligonucleotides, such as chalcone-derivatized oligonucleotides, are incorporated into a nucleic acid aptamer that binds to a specific target, such as a small molecule, such as cocaine, various toxins, or protein targets. In another embodiment, any binding interaction by the derivatized-oligonucleotide (DNA and/or RNA) that increases probe rigidity can be monitored using fluorescence spectroscopy, as the fluorescence of the chalcone increases upon increased rigidity.

Other features and advantages of the present application will become apparent from the following detailed description. It should be understood, however, that the detailed description and the specific examples while indicating preferred embodiments of the application are given by way of illustration only, since various changes and modifications within the spirit and scope of the application will become apparent to those skilled in the art from this detailed description.

### BRIEF DESCRIPTION OF THE DRAWINGS

The present disclosure will now be described in greater detail with reference to the drawings in which:

FIG. 1 is a A) ¹H NMR spectrum and B) ¹³C{¹H} NMR spectrum of a compound in the disclosure.

FIG. 2 is a A) ¹H NMR spectrum and B) ¹³C{¹H} NMR spectrum of a compound in the disclosure.

FIG. 3 is a ³¹P{¹H} NMR spectrum of a compound of the disclosure.

FIGS. 4A-4G are mass spectra of various oligomers incorporating compounds of the disclosure;

FIG. 5 is a normalized fluorescence excitation and emission spectra of probes of the disclosure.

FIG. 6 shows fluorescence emission (A, B, C) and UV-Vis absorbance spectra (D, E, F) displaying hybridization response of oligomers of the disclosure.

FIG. 7 shows a normalized fluorescence excitation and emission spectra (A) of and the response of Th6HI (λₑₓ=425 nm) to duplex formation when hybridized with a complement strand containing Ind6HI (B).

FIG. 8 shows a fluorescence emission spectra displaying the ratiometric response of Narl (5 μM) labelled with an oligomer of the disclosure to hybridization across from the prototypical abasic site (THF).

### DESCRIPTION OF VARIOUS EMBODIMENTS

### Definitions

Unless otherwise indicated, the definitions and embodiments described in this and other sections are intended to be applicable to all embodiments and aspects of the present application herein described for which they are suitable as would be understood by a person skilled in the art.

As used in this application and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “consisting” and its derivatives as used herein are intended to be closed terms that specify the presence of the stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

As used in the present application, the singular forms “a”, “an” and “the” include plural references unless the content clearly dictates otherwise. For example, an embodiment including “a compound” should be understood to present certain aspects with one compound, or two or more additional compounds.

In embodiments comprising an “additional” or “second” component, such as an additional or second compound, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present. The term “and/or” with respect to enantiomers, prodrugs, salts and/or solvates thereof means that the compounds of the application exist as individual enantiomers, prodrugs, salts and hydrates, as well as a combination of, for example, a salt of a solvate of a compound of the application.

The term “suitable” as used herein means that the selection of the particular group or moiety or conditions would depend on the specific steps to be performed, the identity of the components to be transformed and/or the specific use for the compositions, but the selection would be well within the skill of a person trained in the art

The term “C₁₋ₙalkyl” as used herein means straight or branched chain, saturated alkyl groups containing from one to n carbon atoms and includes (depending on the identity of n) methyl, ethyl, propyl, isopropyl, n-butyl, s-butyl, isobutyl, t-butyl, 2,2-dimethylbutyl, n-pentyl, 2-methylpentyl, 3-methylpentyl, 4-methylpentyl, n-hexyl and the like, where the variable n is an integer representing the largest number of carbon atoms in the alkyl radical.

The term “C₂₋ₙalkenyl” as used herein means straight or branched chain, unsaturated alkyl groups containing from two to n carbon atoms and one to three double bonds, and includes (depending on the identity of n) vinyl, allyl, 2-methylprop-1-enyl, but-1-enyl, but-2-enyl, but-3-enyl, 2-methylbut-1-enyl, 2-methylpent-1-enyl, 4-methylpent-1-enyl, 4-methylpent-2-enyl, 2-methylpent-2-enyl, 4-methylpenta-1,3-dienyl, hexen-1-yl and the like, where the variable n is an integer representing the largest number of carbon atoms in the alkenyl radical.

The term “C₂₋ₙalkynyl” as used herein means straight or branched chain, unsaturated alkyl groups containing from two to n carbon atoms and one to three triple bonds, and includes (depending on the identity of n) ethynyl, propynyl, 2-methylprop-1-ynyl, but-1-ynyl, but-2-ynyl, but-3-ynyl, 3-methylbut-1-ynyl, 2-methylpent-1-ynyl, 4-methylpent-1-ynyl, 4-methylpent-2-ynyl, 4-methylpent-2-ynyl, penta-1,3-diynyl, hexyn-1-yl and the like, where the variable n is an integer representing the largest number of carbon atoms in the alkynyl radical.

The term “cycloalkyl” as used herein refers to an aliphatic ring system having 3 to “n” carbon atoms including (depending on the identity of n), but not limited to, cyclopropyl, cyclopentyl, cyclohexyl, and the like, where the variable n is an integer representing the largest number of carbon atoms in the cycloalkyl radical.

The term “alkoxy group” as used herein refers to an “O-alkyl” group wherein “alkyl” is defined herein.

The term “halo” as used herein means halogen and includes chlorine, bromine, iodine and fluorine.

The term “chalcone moiety” as used herein refers to an α,β-unsaturated ketone that is produced when a reactive aldehyde compound is condensed with the compound of formula (I) or (II).

The term “reactive aldehyde compound” as used herein refers generally to a compound having an aldehyde functionality which can participate in an aldol condensation reaction with a compound of Formula (I) or (II).

The term “leaving group” as used herein refers to a group capable of being displaced from a molecule or compound when the molecule or compound undergoes a reaction with a nucleophile.

The term “control” as used herein refers to a sample that has a particular level of fluorescence intensity. A control may contain a known fluorescence intensity and would be a positive control. The control can also be a predetermined standard.

### Compounds of the Disclosure

The present disclosure relates to compounds of Formula (I) which are incorporated into DNA for on-DNA aldehyde capture.

In one embodiment, the compound of the Formula (I) has the structure

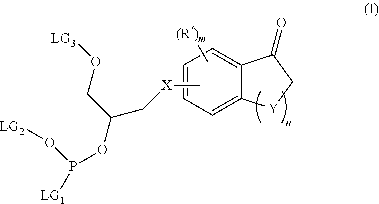

wherein
X is O or NR₁, wherein R₁ is H or (C₁-C₆)-alkyl;
Y is —C(R)(R)—, O, S, or NR₂, wherein R₂ is H or (C₁-C₆)-alkyl;
each R is independently or simultaneously H, halo, (C₁-C₆)-alkyl or (C₁-C₆)-alkoxy;
R′ is a substituent on the benzene ring and is halo, (C₁-C₆)-alkyl or (C₁-C₆)-alkoxy, and m is 0, 1, 2 or 3;
LG₁, LG₂ and LG₃ are each suitable leaving groups;
n is the integer 1, 2 or 3; and
any stereoisomers thereof.

In one embodiment, X is O or NH. In another embodiment, X is O.

In another embodiment, Y is —C(R)(R).

In another embodiment, each R is independently or simultaneously H, halo, or (C₁-C₆)-alkyl. In another embodiment, each R is independently or simultaneously H, halo, or (C₁-C₃)-alkyl. In another embodiment, each R is H.

In a further embodiment, each R′ is independently or simultaneously H, halo, or (C₁-C₆)-alkyl. In another embodiment, each R′ is independently or simultaneously H, halo, or (C₁-C₃)-alkyl. In another embodiment, each R′ is H.

In another embodiment of the disclosure, the groups LG₁, LG₂, and LG₃, are suitable leaving groups for oligomeric nucleic acid synthesis. In one embodiment, the nucleic acid is DNA or RNA.

In another embodiment of the disclosure, LG₁ is

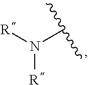

wherein
each R″ is independently or simultaneously (C₁-C₁₀)-alkyl, (C₂-C₁₀)-alkenyl, (C₂-C₁₀)-alkynyl, or (C₃-C₁₀)-cycloalkyl, in which one or more carbon atoms in the alkyl, alkenyl, alkynyl, or cycloalkyl groups can optionally be replaced with an oxygen atom, or NR₁ group.

In another embodiment, each R″ is (C₁-C₃)-alkyl.

In another embodiment, LG₁ is

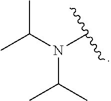 [SMILES: *N(C(C)C)C(C)C]

In another embodiment of the disclosure, LG₂ is

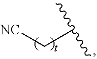

wherein
t is an integer from 1 to 6.

In one embodiment, t is 1 or 2.

In another embodiment, LG₂ is

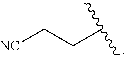 [SMILES: *CCC#N]

In another embodiment of the disclosure, LG₃ is

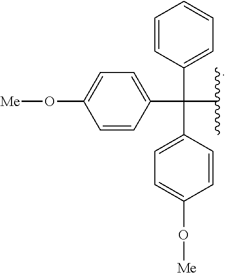 [SMILES: *C(c1ccccc1)(c1ccc(OC)cc1)c1ccc(OC)cc1]

In another embodiment, LG₃ is

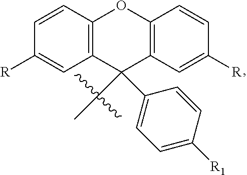

wherein
R is H or (C₁-C₁₀)-alkyl;
R₁ is H, (C₁-C₁₀)-alkyl, halo or NO₂.

In another embodiment of the disclosure, the compound of Formula (I) is

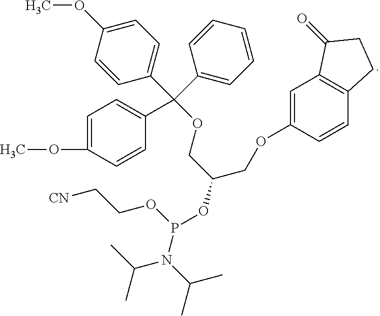

In another embodiment of the disclosure, the compounds of Formula (I) are incorporated or condensed into a nucleic acid. In another embodiment, the present disclosure includes compounds of the Formula (II)

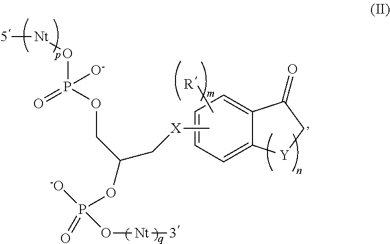

wherein,
X is O or NR₁, wherein R₁ is H or (C₁-C₆)-alkyl;
Y is —C(R)(R)—, O, S, or NR₂, wherein R₂ is H or (C₁-C₆)-alkyl;
each R is independently or simultaneously H, halo, (C₁-C₆)-alkyl or (C₁-C₆)-alkoxy;
R′ is a substituent on the benzene ring and is halo, (C₁-C₆)-alkyl or (C₁-C₆)-alkoxy, and m is 0, 1, 2 or 3;
n is the integer 1, 2 or 3;
wherein Nt is a nucleotide;
p and q are independently an integer between 0 and 1000; and
any stereoisomers thereof.

In another embodiment, the variables X, R₁, Y, R, R₂, R′, m and n are as defined in any of the embodiments of the disclosure.

In another embodiment, the nucleotide, which is composed of a phosphate group, a 5-carbon sugar, and a nitrogenous base, which may be cytosine (C), thymine (T), uracil (U), adenine (A), guanine (G) and derivatives thereof.

In another embodiment, p and q are independently integers between 1 and 1000, or 1 and 500, or 1 and 250, or 1 and 100, or 1 and 50, or 1 and 10.

In another embodiment, the compounds of Formula (II) are oligomers having compounds of the Formula (II) condensed within the nucleic acid chain.

In another embodiment, the oligomer compound of the Formula (II) has the structure

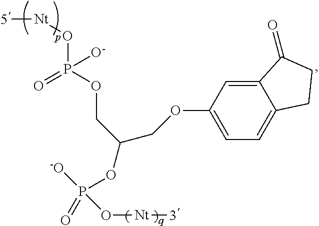

wherein Nt, p and q are as defined above in any embodiment.

In another embodiment of the disclosure, the compounds of Formula (II) are reacted with reactive aldehyde compounds, such as aromatic aldehydes having exocyclic amino or internal nitrogen donor groups, resulting in compounds of the Formula (III) which are fluorescent molecular rotors. Examples of reactive aldehydes with exocyclic amino and intemal nitrogen donor groups:

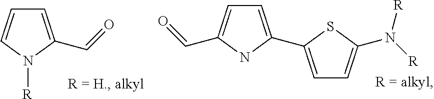

wherein R is H or (C₁-C₁₀)-alkyl;

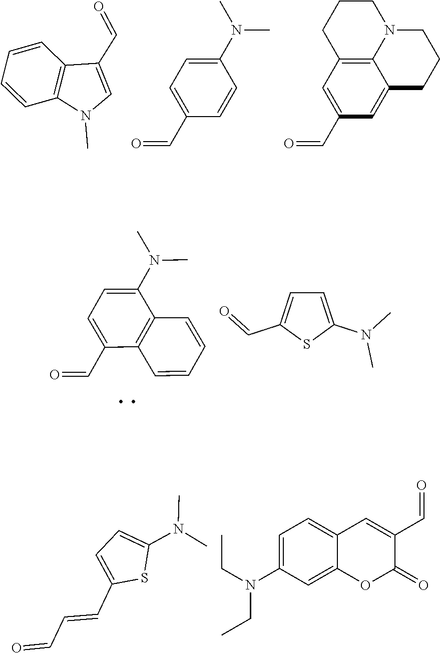

In another embodiment, the reactive aldehydes are aromatic groups containing phenolic groups. Examples of aromatic aldehydes containing phenolic (OH) groups attached to the aromatic ring:

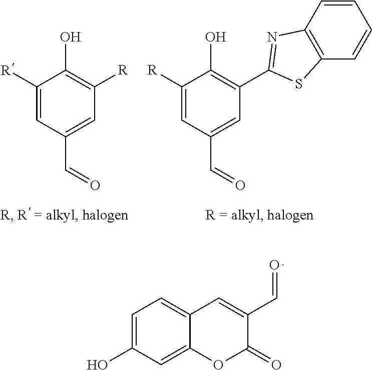

In another embodiment, the compound comprising a reactive aldehyde reacts in an aldol condensation reaction at the α-position of the ketone of the compound of Formula (II) to obtain compounds of the Formula (III)

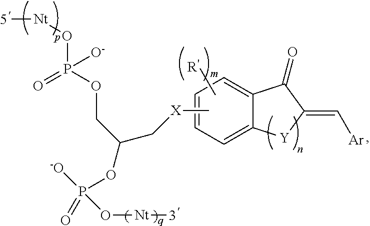

wherein,
Ar is an aromatic moiety; and
the variables X, R₁, Y, R, R₂, R′, m and n are as defined in any of the embodiments of the disclosure.

In one embodiment, the compound of Formula (III), after being reacted with the reactive aldehyde compound, contains a chalcone moiety as shown below

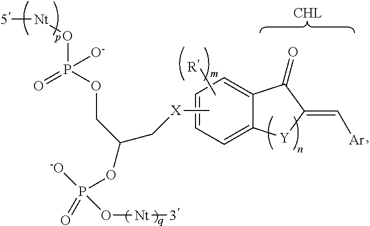

wherein CHL is a chalcone moiety and Ar is an aromatic moiety.

In another embodiment, the aromatic group (Ar) present in the compound of Formula (III) is the moiety attached to the reactive aldehyde moiety in the reactive aldehyde compound. For example, in the compounds below, the reactive aldehyde moiety and aromatic “R” group moiety are shown below:

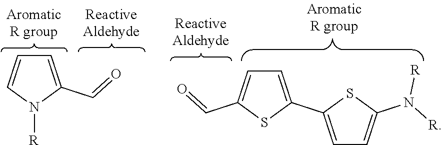

In one embodiment, the compound of Formula (III) has the structure

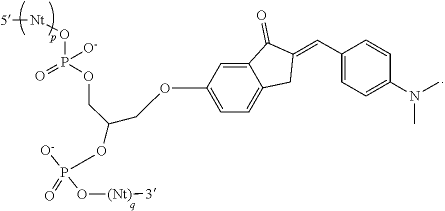

### Methods of the Disclosure

The compounds of the disclosure, and in particular, the compounds of the Formula (III) are useful as, for example, fluorescent molecular rotors. In particular, the compounds can be used as fluorescent probes for detecting and sensing the structure, dynamics and binding interactions of nucleic acids. For example, the oligonucleotide containing the probe (compound of Formula (III)) is paired with a complementary nucleic acid strand to produce the duplex nucleic acid (double stranded). In one embodiment, the probe fluorescence intensity increases upon hybridization because of increased rigidity of the probe within the duplex structure.

Accordingly, in one embodiment of the disclosure, there is included a method for the detection of nucleic acid hybridization to create duplex nucleic acids (DNA and/or RNA) comprising,
a) contacting an oligonucleotide having the structure of Formula (II) with a reactive aldehyde compound to obtain a chalcone-derivatized oligonucleotide of the Formula (III);
b) contacting the chalcone-derivatized oligonucleotide with a complementary nucleic acid (DNA and/or RNA) through hybridization to afford a duplex nucleic acid structure; and
c) measuring an increase of fluorescence intensity of the duplex nucleic acid structure indicating nucleic acid hybridization.

In another embodiment of the disclosure, there is included a method for the detection of nucleic acid hybridization to create duplex nucleic acids (DNA and/or RNA) comprising,
a) contacting a chalcone-derivatized oligonucleotide with a complementary nucleic acid (DNA and/or RNA) through hybridization to afford a duplex nucleic acid structure; and
b) measuring an increase of fluorescence intensity of the duplex nucleic acid structure indicating nucleic acid hybridization.

In another embodiment, there is included a method of detecting a nucleic acid, comprising
a) contacting a sample that contains complementary DNA or RNA with a chalcone derivatized oligonucleotide having the structure of Formula (III);
b) detecting a fluorescence signal at a wavelength specific for the chalcone moiety;
c) comparing the fluorescence signal of (b) with the fluorescence intensity of a control sample, which lacks complementary DNA or RNA, or simply lacks nucleic acids;
   wherein detection of a signal in the sample having a fluorescence intensity greater than the control sample indicates the sample contains the complementary nucleic acid for Formula (III) to produce a duplex structure.

In one embodiment, the method is used to detect RNA in the cytoplasm of cells.

It will be understood that the increase in fluorescence intensity depends on the nucleic acid concentration in the sample. For quantification, a calibration curve is generated based on the nucleic acid of known concentration which is used to compare the fluorescence intensity signal from the sample under investigation

In another embodiment, derivatized oligonucleotides, such as chalcone-derivatized oligonucleotides, are incorporated into a nucleic acid aptamer that binds to a specific target, such as a small molecule, such as cocaine, various toxins, or protein targets. In another embodiment, any binding interaction by the derivatized-oligonucleotide (DNA and/or RNA) that increases probe rigidity can be monitored using fluorescence spectroscopy, as the fluorescence of the chalcone probe increases upon increased rigidity.

Accordingly, in another embodiment, there is included a method of detecting a target, comprising
a) contacting a sample with a chalcone derivatized aptamer having the structure of Formula (III), wherein the aptamer binds the target;
b) detecting a fluorescence signal at a wavelength specific for the chalcone moiety;
c) comparing the fluorescence signal of (b) with the fluorescence intensity of a control sample;
   wherein detection of a signal in the sample having a fluorescence intensity greater than the control sample indicates the sample contains the target.

In one embodiment, the sample matrix is a biological sample such as saliva, blood, or urine, or an environmental sample.

In another embodiment, the target is cocaine, a toxin or any other protein target.

Although the disclosure has been described in conjunction with specific embodiments thereof, if is evident that many alternatives, modifications and variations will be apparent to those skilled in the art. Accordingly, it is intended to embrace all such alternatives, modifications and variations that fall within the spirit and broad scope of the appended claims. In addition, citation or identification of any reference in this application shall not be construed as an admission that such reference is available as prior art to the present disclosure.

### EXAMPLES

The operation of the disclosure is illustrated by the following representative examples. As is apparent to those skilled in the art, many of the details of the examples may be changed while still practicing the disclosure described herein.

### Materials and Methods

Unmodified oligonucleotides, enamine catalysts, 6-hydroxy-indanone, (R)-(+)-glycidol, DNA synthesis materials, solvents and all other reagents were purchased from commercial sources and used as received excluding a few select aldehydes, which were synthesized via known procedures (7-diethylaminocoumarin-3-aldehyde (Cou), 3-(5-(Dimethylamino)thiophen-2-yl)acrylaldehyde (Eth), and 3-dimethylaminonapthalene-1-carbaldehyde(Nap)).^{[1-3]} Buffers were prepared from their respective salts using water from a filtration system (18.2 MΩ) with neutralization to the desired pH using 5M aq. HCl/NaOH. Modified oligonucleotides were synthesized on a 1 μmol scale with DMT-ON protection using an ABI394 DNA/RNA synthesizer with trityl monitor. Purification of oligos after solid-phase synthesis and on-strand aldol was performed using Glen-Pak DNA purification cartridges or RP-HPLC on an Agilent 1200 series HPLC system equipped with a diode array detector, fluorescence detector, autosampler, and fraction collector. Ultraviolet-visible (UV-vis) spectra were obtained on a Cary 300-Bio UV-vis spectrophotometer equipped with a 6×6 multicell block Peltier temperature control unit. Fluorescence measurements were acquired on either a Cary Eclipse Fluorescence spectrophotometer or an Edinburgh spectrofluorometer FS5 at ambient temperature. CD spectra were obtained on a Jasco J-815 CD spectrophotometer using quartz cells (110-QS) with a light path of 1 mm and monitored between 200 and 400 nm at a bandwidth of 1 nm and a scanning speed of 100 nm/min at ambient temperature. NMR spectra were recorded on Bruker Avance 300 or 400 MHz spectrometers at room temperature. Low-resolution mass spectra were acquired on a LTQ XL lon Trap mass spectrometer using an electrospray ionization source ESI.

### Solid Phase DNA Synthesis

Solid-phase DNA synthesis of 6HI modified Narl utilized standard and 6HI modified phosphoramidites with standard synthesis reagents. The 6HI-phosphoramidite was inserted into the G3 (X) position of the 12mer Narl sequence (5′-CTCGGCXCCATC-3′) with standard coupling times for all modified and unmodified phosphoramidites. Post-synthesis, oligos containing the 5′DMT-ON protection was deprotected in 1 mL of 30% ammonium hydroxide for 24 h at room temperature. Crude oligos were the purified using Glen-Pak DNA purification cartridges. Cartridge eluent purity was verified via RP-HPLC, and the solvent mixture was concentrated to ˜1 mM using a ThermoSavant DNA 120 SpeedVac at a medium drying rate with quantification via UV-Vis measurements at 260 nm. Prepared oligo mixtures were then used in downstream on-strand aldol condensations with storage at 4° C.

### On-Strand Aldol Reactions

Aldol condensations with 6HI-labeled DNA substrates were performed via one general catalytic method, with changes of co-solvent being the only modification. For each reaction, aldehydes were prepared in DMSO (except for Cou (0.25 M) and Ju (0.75 M), which were dissolved in DMF) to a concentration of ˜1 M. 35 μL of the DMSO/DMF stock solution was then added to 50-60 μL of the 6HI modified Narl oligo (1 mM) which was dissolved in MQ H2O to give an approximately 30:70 DMSO/H2O mixture. Reaction mixture at this point typically contained some quantity of precipitated aldehyde. Piperidine (3.5 μL) was then added to the reaction mixture followed by subsequent heating at 75° C. with mixing via vortex every 1-2 hours. Aldehyde underwent solvation readily upon heating. After 4-6 h, the reaction mixture was cooled to room temperature and 5% (V/V) 5 M NaCl was added along with 3× the volume of absolute ethanol. The mixture was stored in the freezer at −20° C. overnight. After centrifugation for 1 h, the supernatant was decanted, and the DNA pellet was air-dried. The pellet was redissolved in purified 18.2 MΩ water and purified by HPLC that was carried out at 70° C. using a 5 μm reversed phase semipreparative C18 column (100×10 mm²) with a flow rate of 3.3 mL/min in various gradients of buffer A in buffer B (buffer A=30:70 aqueous 50 mM TEAA, pH 7.2/acetonitrile; buffer B=95:5 aqueous 50 mM TEAA, pH 7.2/acetonitrile). Peaks showing high absorbance at both 254 nm (DNA) and 430-60 nm (chalcone modification) were collected. Yields were estimated using the relative integrals of product and reactant DNA peaks. Following purification, the collected samples were lyophilized to dryness and redissolved in 200 μL of 18.2 MΩ water. Samples were subsequently quantified using UV-vis measurements and analyzed by ESI-MS (see the SI for ESI-MS spectra and HPLC chromatograms.

### Thermal Melting and Spectroscopic Measurements

All fluorescent/UV-vis spectra and thermal melting measurements (Tₘ) were carried out using a 10 mm light path quartz glass cells with a baseline correction. For melting temperature measurements with Narl substrates, DNA stock solutions were diluted to 5 μM in binding buffer (50 mM Na2PO4 buffer with 0.1 M NaCl (pH 7)) to which 1 equivalent of complementary strand with either the native cytosine or abasic THF modification opposite the dye was added. UV absorbance was monitored at 260 nm as a function of temperature with five alternating ramps from 10-90 and 90-10° C. at a heating/cooling rate of 0.5° C./min. Average Tₘ values were calculated using hyperchromicity calculations performed with the Varian Thermal melting software. Relative fluorescent quantum yields (Φ_{fl}) were measured at three different concentrations for each probe in the full duplex using either fluorescein in 0.1M NaOH or Rhodamine 101 in absolute ethanol as fluorescent standards.

### Example 1—Phosphoramidite Synthesis

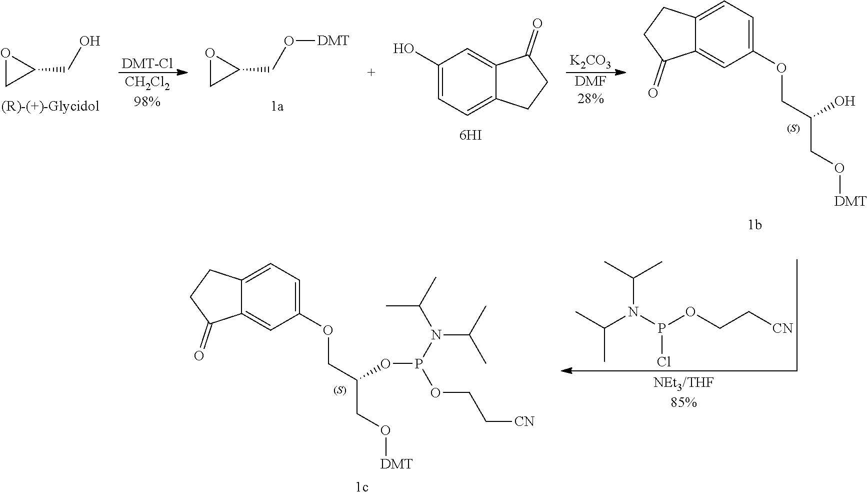

### Synthesis of (S)-6-hydroxy-indanone phosphoramidite (1c)

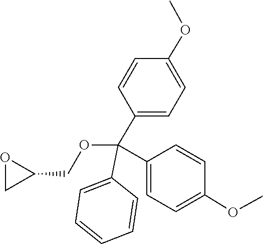 [SMILES: COc1ccc(C(OC[C@@H]2CO2)(c2ccccc2)c2ccc(OC)cc2)cc1]

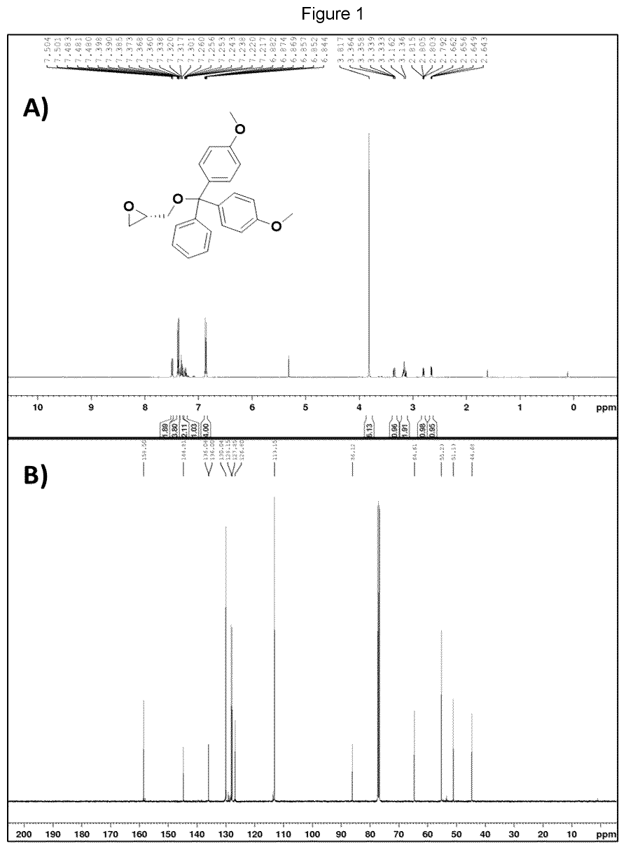

Glycidol-DMT (1a): Compound 1a was synthesized according to literature procedure.^{[4]} (R)-glycidol (1.00 mL, 15.1 mmol, 1.00 eq.) was added to an oven-dried round bottom flask followed by stirring under argon in 5 mL dry DCM and the addition of freshly distilled triethylamine (8.00 mL, 57.4 mmol, 3.82 eq.). DMT-Chloride solution (20 mL, 0.88 M, 17.67 mmol, 1.17 eq.) in DCM was added dropwise at 0° C. followed by an additional 10 mL DCM to promote stirring of the solution. Reaction mixture was stirred at room temperature for 16 hours under argon followed by washing with half saturated aq. NaHCO₃. Organic extraction was carried out using DCM (1×60 mL) followed by drying over MgSO₄ and removal of solvent under reduced pressure to give a dark red residue. Residue was then purified using silica gel chromatography (99:1, dichloromethane:triethylamine) to provide 1a as a colourless viscous oil (5.5838 g, 98% yield). ¹H NMR (CDCl₃, 400 MHz): δ 7.51-7.47 (m, 2H), 7.41-7.35 (m, 4H), 7.35-7.28 (m, 2H), 7.27-7.20 (m, 1H), 6.89-6.84 (m, 4H), 3.82 (s, 6H), 3.50 (dd, 1H, J=9.9 Hz, 2.3 Hz), 3.20-3.10 (m, 2H), 2.82-2.78 (m, 1H), 2.68-2.63 (m, 1H). ¹³C{1H} NMR (CDCl₃, 100 Hz) δ 158.5, 144.8, 136.0, 130.0, 128.2, 127.9, 126.8, 113.1, 86.1, 64.6, 55.2, 51.1, 44.7. MS (ESI) m/z: [M+Na]⁺ calcd for C₂₄H₂₄O₄=399.16, found 398.94. (FIG. 1 shows the NMR of the 1a).

### DMT Protected (S)-6-hydroxy-indanone glycerol nucleoside (1b)

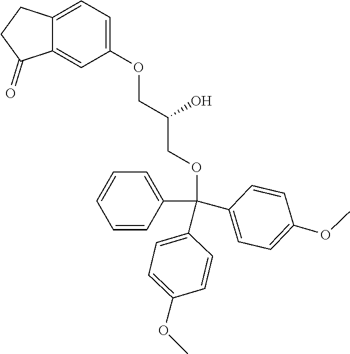 [SMILES: COc1ccc(C(OC[C@@H](O)COc2ccc3c(c2)C(=O)CC3)(c2ccccc2)c2ccc(OC)cc2)cc1]

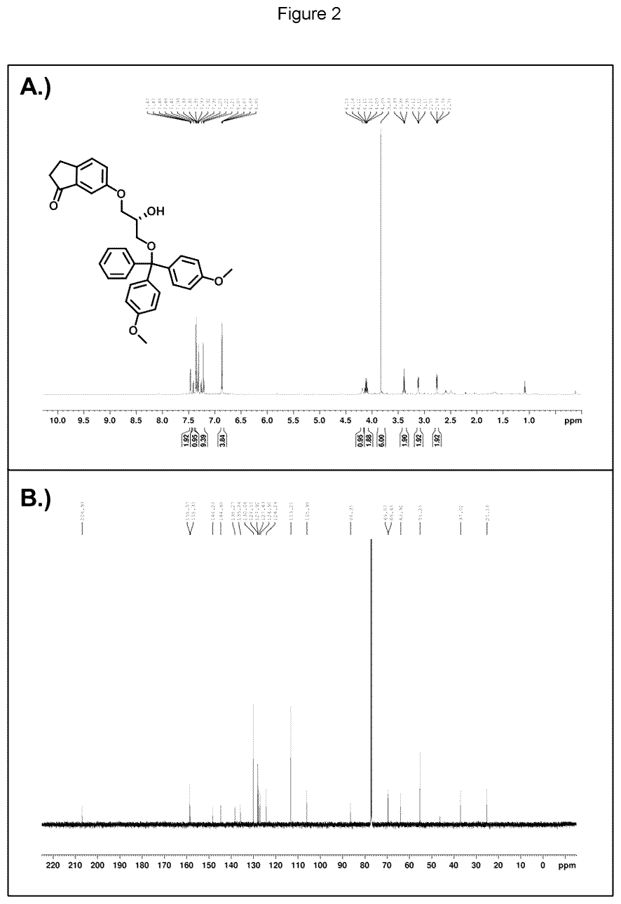

6-hydroxy-indanone (1.1775 g, 7.94 mmol, 1.00 eq.), 1a (3.3618 g, 8.95 mmol, 1.12 eq.) and K₂CO₃ were combined 30 mL DMF and heated at 90° C. via oil bath for 48 hours. Reaction mixture was then cooled to room temperature and diluted with 75 mL EtOAc followed by washing of the organic layer with deionized water (3×125 mL) and 5% LiCl (1×100 mL). Organic layer was then filtered through celite and dried over Na₂SO₄. Solvent was then removed under reduced pressure to give an oily residue which was purified via silica gel chromatography (29.5:69.5:1 ethyl acetate:hexanes:triethylamine) to give 1b as a foamy white solid (1.1563 g, 28%). ¹H NMR (CDCl₃, 400 MHz): 7.48-7.44 (m, 2H), 7.40 (d, 1H, J=8.5 Hz), 7.37-7.34 (m, 4H), 7.33-7.29 (m, 2H), 7.27-7.24 (m, 1H), 7.23-7.19 (m, 2H), 6.88-6.84 (m, 4H), 4.18 (q, 1H, J=4.6 Hz), 4.14-4.07 (m, 2H), 3.83 (s, 6H), 3.41-3.35 (m, 2H), 3.11 (t, 2H, J=5.6 Hz), 2.77-2/74 (m, 2H). ¹³C{1H} NMR (CDCl₃, 100 Hz) δ 206.9, 158.6, 158.4, 148.3, 144.7, 138.3, 135.8, 130.0, 128.1, 127.9, 127.4, 126.9, 124.2, 113.2, 106.0, 86.3, 69.6, 69.5, 63.9, 55.2, 37.0, 25.2. (FIG. 2 shows the NMR of 1b).

### (S)-6-hydroxy-indanone phosphoramidite (1c)

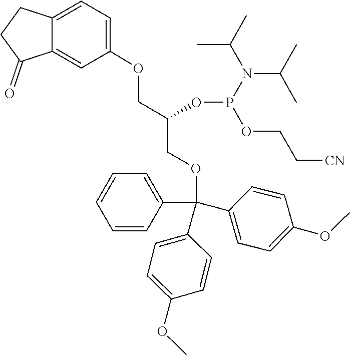 [SMILES: COc1ccc(C(OC[C@H](COc2ccc3c(c2)C(=O)CC3)OP(OCCC#N)N(C(C)C)C(C)C)(c2ccccc2)c2ccc(OC)cc2)cc1]

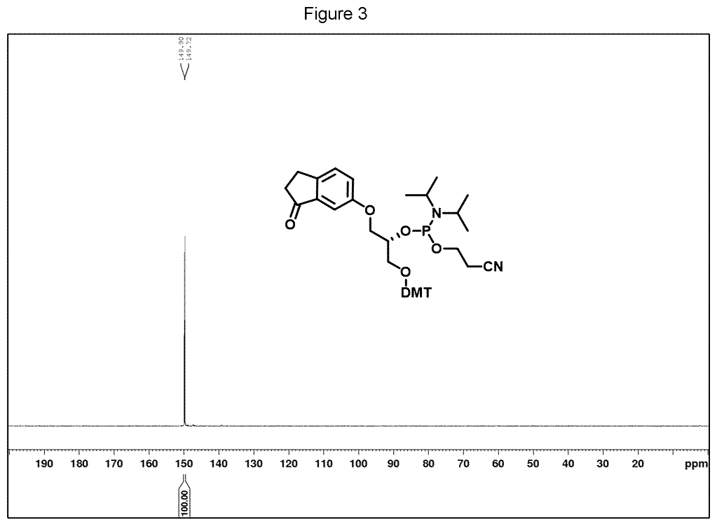

1b (0.2844 g, 0.54 mmol, 1.00 eq) was placed in an oven dried round bottom flask and co-evaporated with dry toluene (3×8 mL) and followed by solvation in dry THF (8 mL) and the addition of freshly distilled TEA (0.32 mL, 2.29 mmol, 4.25 eq). Reaction mixture was then stirred under argon for 10 minutes followed by the dropwise addition of 2-cyanoethyl N,N-diisopropylchlorophosphoramidite (0.20 mL, 0.89 mmol, 1.65 eq.) and stirring for a further 2 hours. Solvent was then removed under reduced pressure and organic layer was extracted via the addition of ethyl acetate containing 3% triethylamine. Organic layer was then washed with half sat. NaHCO₃ (2×50 mL) followed by brine (1×30 mL). Solvent was then removed under reduced pressure and the residue was purified via silica gel chromatography (39:59:2 ethyl acetate:hexanes:triethylamine) to afford product 1c (330 mg, 85%) which was characterized via ³¹P NMR and stored under argon at −20° C. ³¹P NMR (162 MHz, CDCl3): δ 149.90, 149.72. FIG. 3 shows the ³¹P NMR of 1c.

### Example 2—Incorporation of 1c into Oligonucleotides

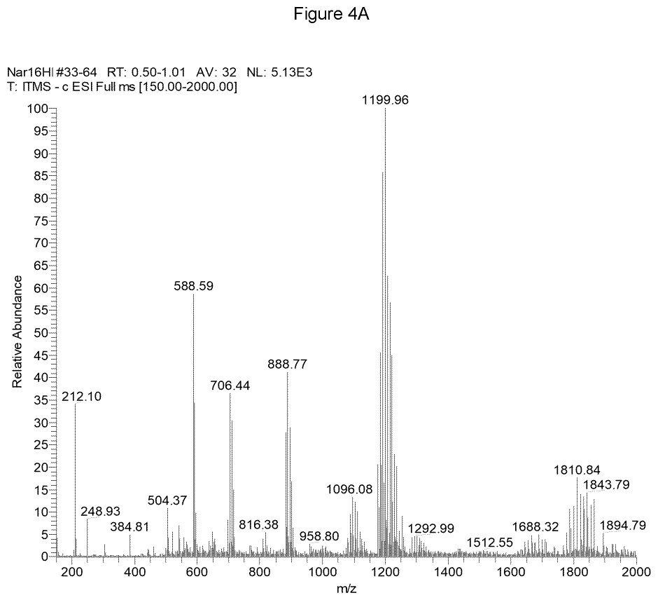

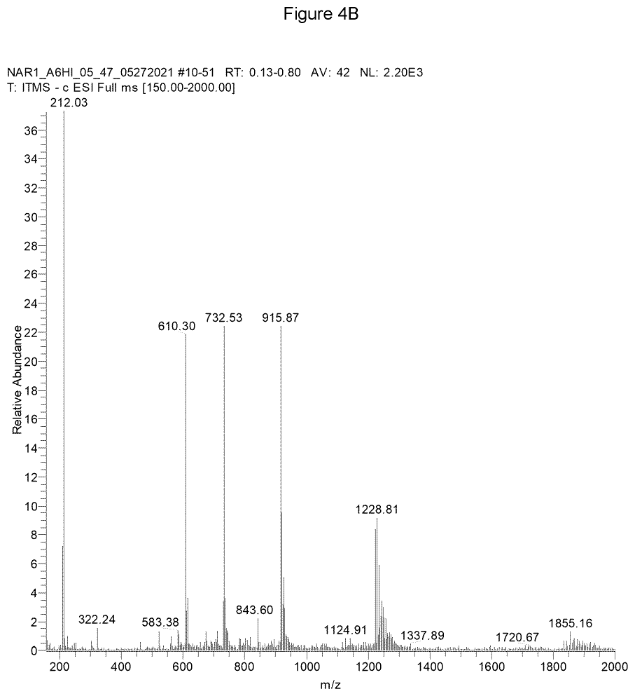

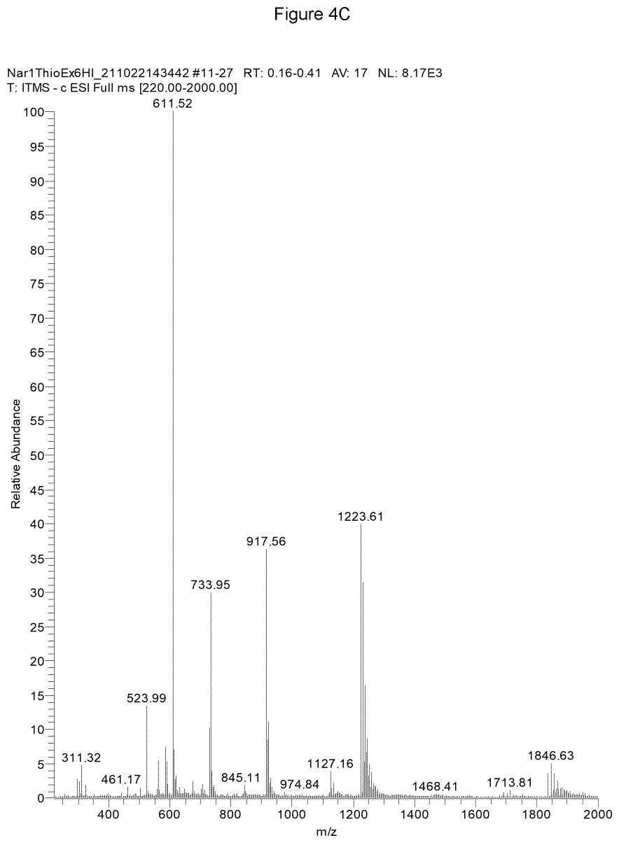

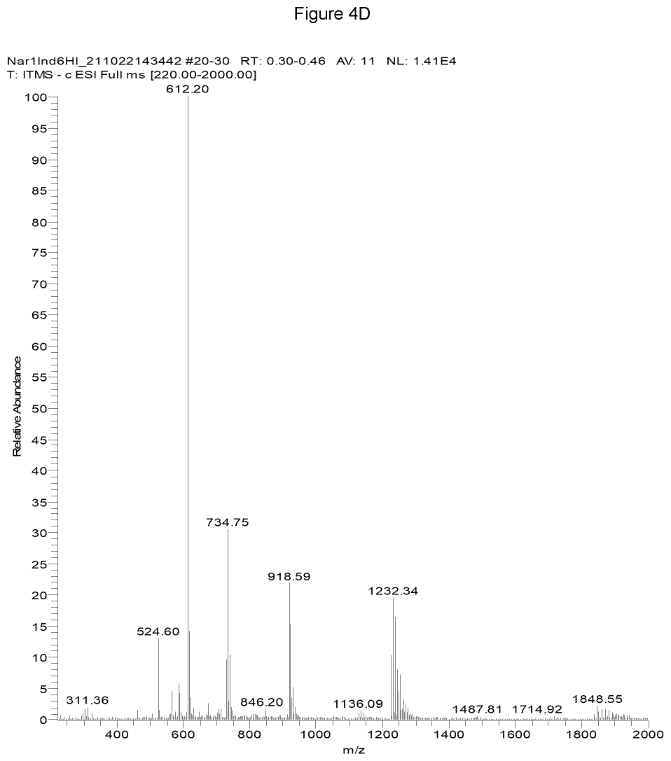

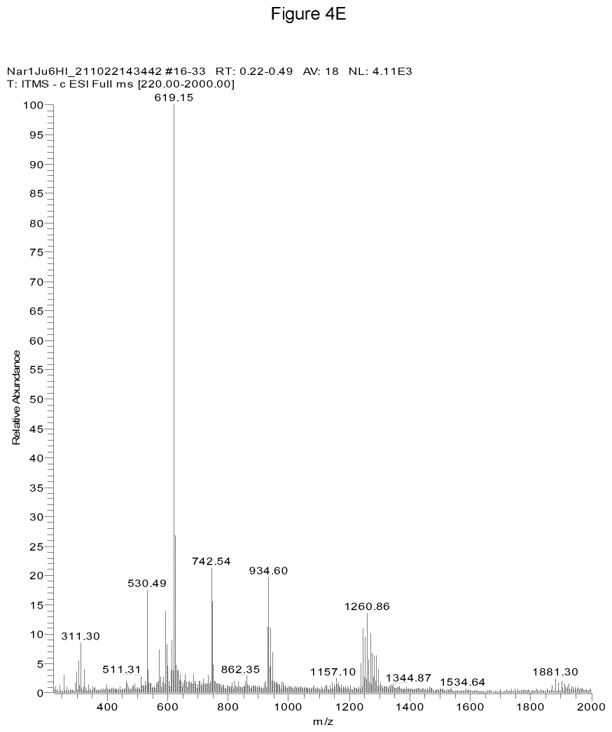

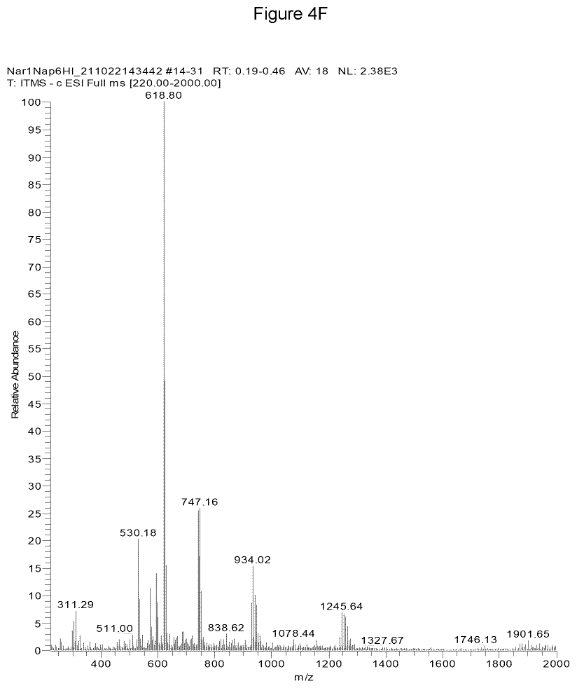

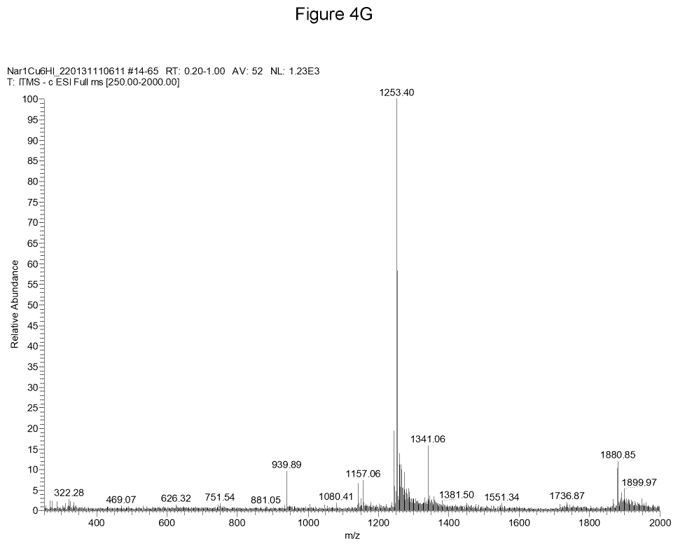

Synthesis of the 6HI phosphoramidite was easily accessible (2 steps from 6HI and the known DMT-(R)-(+)-glycidol, and compatible with standard solid-phase oligonucleotide synthesis (coupling times, reagents and deprotection using NH₄OH). The 6HI phosphoramidite was inserted into the G3-site (X) of Narl (5′-CTC-G₁G₂C-X-CCA-TC-3′), which was previously utilized as an oligonucleotide substrate for 3FI and 4BrA, permitting direct comparison of probe performance. Conditions for the 6HI on-strand aldol condensations mirrored those which were presented with 3FI. Most reactions followed the same general template; 50 μL of 6HI-labelled Narl was prepared to a stock concentration of ˜1 mM and combined with 35 μL of a 1M DMSO stock of the respective aldehyde. To this oligo/aldehyde mixture was added 3.5 μL piperidine followed by heating at 75° C. for 3-6 hours and subsequent ethanol precipitation of the oligonucleotide mixture. Reaction yields were obtained via high performance liquid chromatography (HPLC) and ranged from 35-97%, as shown in Table 1. Mass spectrometry was used to confirm the identity of all purified oligos as shown in FIG. 4A-4G.

TABLE S1
Yields and MS Analysis of Modified Narl Oligonucleotides.

| | | | | ExptI | |
|---|---|---|---|---|---|
| | | | Calc. | (m/z) | ExptI |
| Narl^{a} | Yield ^{b} | Formula | Mass^{c} | (ESI⁻)^{d} | Mass |
| 6HI | / | C₁₁₆H₁₄₈N₃₇O₇₁P₁₁ | 3536.63 | [M − 6H]⁶⁻ = | 3537.54 |
| | | | | 588.59 | |
| An6HI | 45 | C₁₂₅H₁₅₇N₃₈O₇₁P₁₁ | 3667.70 | [M − 6H]⁶⁻ = | 3667.80 |
| | | | | 610.30 | |
| Th6HI | 35 | C₁₂₃H₁₅₅N₃₈O₇₁P₁₁S | 3673.66 | [M − 4H]⁴⁻ = | 3674.24 |
| | | | | 917.56 | |
| Ind6HI | 97 | C₁₂₆H₁₅₅N₃₈O₇₁P₁₁ | 3677.68 | [M − 6H]⁶⁻ = | 3679.20 |
| | | | | 612.20 | |
| Ju6HI | 35 | C₁₂₉H₁₆₁N₃₈O₇₁P₁₁ | 3719.73 | [M − 6H]⁶⁻ = | 3720.90 |
| | | | | 619.15 | |
| Nap6HI | 40 | C₁₂₉H₁₅₉N₃₈O₇₁P₁₁ | 3717.71 | [M − 6H]⁶⁻ = | 3718.80 |
| | | | | 618.80 | |
| Cou6HI | 31 | C₁₃₀H₁₁₆N₃₈O₇₃P₁₁ | 3763.72 | [M − 3H]³⁻ = | 3763.20 |
| | | | | 1253.4 | |
| ETh6HI | 63 | | | | |

^{a}Modification at G3 (X) of the 12mer Narl oligonucleotide (5′-CTC-GGC-X-CCA-TC-3′).
^{b} Percent yield from integration of HPLC trace assuming the same extinction coefficients for the 6HI labelled Narl precursor and chalcone product, Narl (ε₂₆₀ = 102,100 M⁻¹cm⁻¹).
^{c}Monoisotopic mass of most abundant isotopologue containing one ¹³C isotope.
^{d}Measured m/z from mass spectrum.

Upon successful incorporation of 6HI derived FMRs into Narl, performance was tested through addition of the full NarlComp oligo, where X is the model abasic site, tetrahydrofuran (THF) or C (5′-GAT-GG-X-GCC-GAG-3′). Apart from the ETh probe, the 6HI derived chalcones exhibited minimal impact on duplex stability, with ΔTₘ values ranging from −1.9 to 1.7° C. compared to full length duplex containing a native G:C base pair. Comparatively, probes derived from 3FI and 4BrA (i.e., 4PI and [CHOTh]An, Table 2) strongly decreased duplex stability with ΔTₘ values greater than −10° C.

TABLE 2
Thermal Melting Parameters and Photophysical Properties of Modified Nar Duplexes.

| | Tₘ(ΔTₘ)^{a} | | λₑₓ | εₘₐₓ | λₑₘ | | Δν^{c} | | brightness |
|---|---|---|---|---|---|---|---|---|---|
| Donor | ° C. | | (nm) | (cm⁻¹M⁻¹) | (nm) | Iᵣₑₗ^{b} | (nm) | Φ_{fl}^{d} | (Φ_{fl} · εₘₐₓ) |
| Ind | 65.7 | (1.7) | 450 | 24,300 | 518 | 7 | 68 | 0.09 | 2,187 |
| An | 62.1 | (−1.9) | 475 | 28,500 | 580 | 8 | 105 | 0.27 | 7,695 |
| Ju | 63.4 | (−0.6) | 512 | 23,000 | 610 | 13 | 98 | 0.13 | 2,990 |
| Nap | 63.6 | (−0.4) | 475 | 18,600 | 615 | 75 | 140 | 0.18 | 3,348 |
| Th | 64.4 | (0.4) | 545 | 39,000 | 585 | 2 | 40 | 0.39 | 15,210 |
| ETh | 47.3 | (−16.7) | 581 | 21,000 | 680 | 8 | 99 | 0.08 | 1,680 |
| Cou | 59.6 | (−4.4) | 521 | 21,800 | 612 | 1.5 | 91 | 0.29 | 6,090 |
| 4PI | 51.0 | (−13) | 465 | 28,000 | 530 | 2.8 | 65 | — | — |
| [CHOTh]An | 52.6 | (−11.4) | 444 | — | 565 | 2.2 | 121 | — | — |

^{a}Tₘ values of duplexes (5 μM) measured in 50 mM sodium phosphate buffer, pH 7, with 0.1M NaCl, heating rate of 0.5° C./min, errors are ±1° C. ΔTₘ = Tₘ (modified duplex, X = surrogate opposite THF) − Tₘ (unmodified duplex containing X = G opposite C).
^{b}Iᵣₑₗ = emission intensity of probe in the duplex versus the single-strand.
^{c}Stokes shift = emission maximum − excitation maximum in nm.
^{d}Quantum yields for the probes in the duplex structure

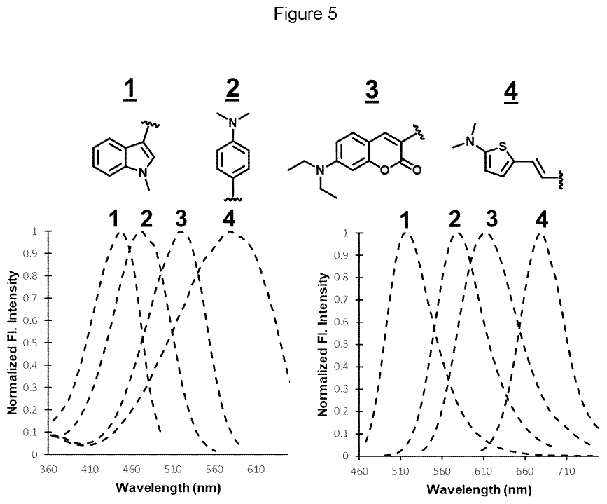

Aldehydes utilized to generate the chalcone library were either commercially available or readily accessible through known reaction chemistries. Absorbance (λₘₐₓ) and emission (λₑₘ) values for the 6HI derived probes proved to be highly sensitive to the nature of the conjugated aromatic amine donor, successfully creating an FMR palette with excitation (450-581 nm) and emission wavelengths (518-680 nm) spanning the visible region (Table 2/FIG. 5). FIG. 5 shows normalized fluorescence excitation and emission spectra of probes 1-4 in the Narl duplex (5 μM) measured in 50 mM sodium phosphate buffer, pH 7, with 0.1 M NaCl at room temperature.

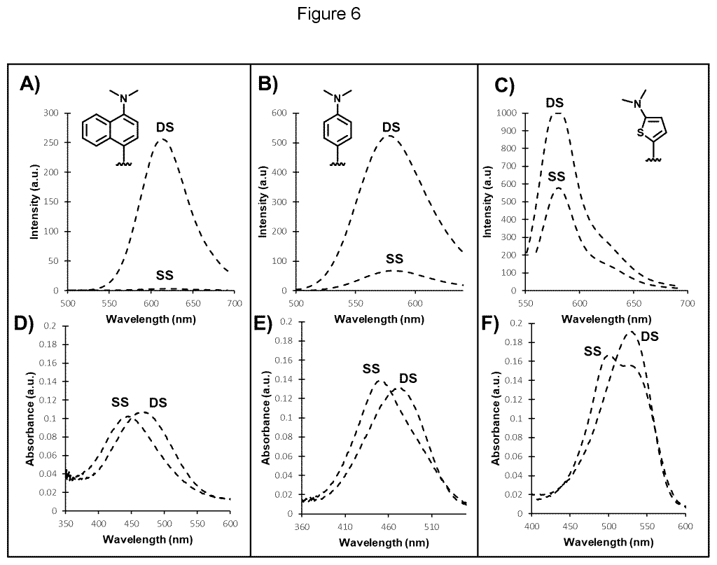

All probes displayed strong bathochromic shifts in their absorbance spectra upon hybridization, as exemplified in FIG. 6 (bottom traces) for three representative probes. Congruent with this bathochromic shift, was a strong increase in fluorescence intensity upon hybridization, with Iᵣₑₗ values ranging from 2-75-fold “turn-on” fluorescence for the duplex (DS) relative to the single strand (SS) (top traces,

FIG. 6). Without being bound by theory, the hybridization induced bathochromic shift/fluorescent enhancement of 6HI FMRs lies in the existence of two different states: stacked vs un-stacked, where un-stacked is almost devoid of fluorescence in the SS. The thiophene (Th6HI) analogue represents the clearest example of this concept (FIG. 3C), where two well defined absorbances were present in the SS (stacked at ˜545 nm vs. unstacked at ˜490 nm). Preferential excitation of the stacked conformation at ˜ 545 nm generated the emission at ˜585 nm. FIG. 6 shows fluorescence emission (A, B, C) and UV-Vis absorbance spectra (D, E, F) displaying hybridization response of Narl (5 μM) when labelled with Nap6HI (A/D), An6HI (B/E), and Th6HI (C/F) across from the prototypical abasic site (THF). Spectra were recorded in 50 mM sodium phosphate buffer, pH 7, with 0.1 M NaCl at room temperature.

It was also observed that the relative size of the aromatic donor rings had an impact on the population of “stacked” vs “un-stacked” states in the SS. The bulkiest donor probe Nap6HI (FIG. 6A), which possesses two aromatic rings, exhibited the least amount of “stacked” structure in the SS, suggesting that the probe is highly twisted. Consequently, this probe can readily produce a twisted-intramolecular charge transfer state (TICT) with strongly quenched emission in the SS. Duplex formation creates a planar “stacked” structure, which inhibits TICT, and the Nap6HI probe exhibited the largest increase in fluorescence intensity upon duplex formation (Iᵣₑₗ=75-fold increase). Removal of an aromatic ring from Nap6HI to afford the An6HI probe increases the donor character and planarity of the ground-state structure to afford a greater degree of the “stacked” conformation in the SS. The donor character of An6H1 is further increased by replacement of the An group with the thiophene ring to afford the Th6HI probe. Thus, these probes exhibit smaller Iᵣₑₗ values (8-fold for An6HI and 2-fold for Th6HI) due to enhanced emission in the SS with increased stacked character.

There was also a steady increase in the molar extinction coefficient (εₘₐₓ) as the ring size decreases. Reasoning for these changes in εₘₐₓ lies in the fact that the larger rings more easily diffuse charge density placed into the aromatic system by the exocyclic amine donor. As such, the relative dipole moments of the ground state fluorophores are decreased with respect to increasing ring size, thus limiting the potential εₘₐₓ. This effect has important implications on probe brightness (B) where B is a direct function of εₘₐₓ. Brightness values increase steadily going from Nap6HI (3,348 cm⁻¹M⁻¹) to An6HI (7,695 cm⁻¹M⁻¹) to Th6HI (15,210 cm⁻¹M⁻¹). Conversely, fluorophore Stokes shifts follow the opposite trend, where they decrease in value going from Nap6HI (140 nm) to An6HI (105 nm) to Th6HI (40 nm). Larger ring sizes (i.e., Nap6HI) allow elimination of background fluorescence, and thus are better suited towards sensing applications where a signal change is required. In comparison, the smaller thiophene ring size within Th6HI allows for superior brightness, which is useful in applications where general tracking of a label is the goal. Intermediate ring sizes (i.e., An6HI) offer a mixture of both superior brightness and strong signaling, highlighting the utility of this modular approach.

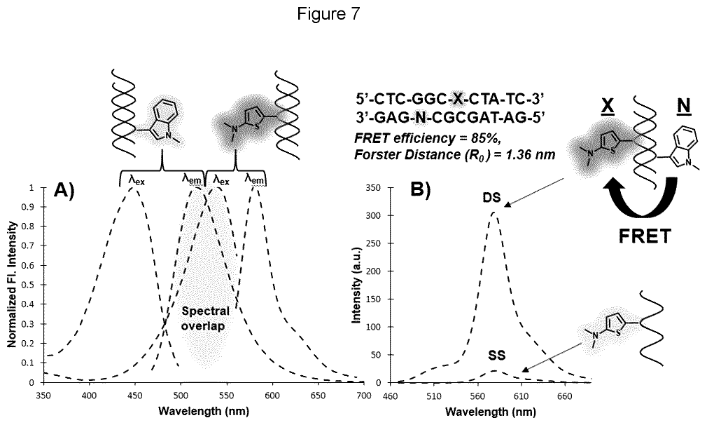

The design of such a modular approach also permitted the development of internal FRET pairs which can be used to both decrease background and increase signal upon hybridization. Typically, FRET pairs in duplex environments are composed of end-labeled platforms where commercially available dyes are incorporated at both the 3′ and 5′-ends.^{26,27} Although 5′-end labelling is typically straightforward, the directionality of standard phase oligonucleotide synthesis from 3′ to 5′ can cause issues with incorporation at the 3′-end. Additionally, placement of dyes directly across from one another can cause dye-dye interactions that quench fluorescence.¹⁰ Utilizing 6HI as the synthetic platform, a FRET pair was generated via placement of an acceptor (Th6HI) and donor (Ind6HI) into complementary strands, with separation by 3 base pairs. At this distance of separation, a FRET efficiency of 85% was observed which correlates to a Förster distance (R₀) of ˜1.36 nm. Additionally, this FRET platform was able to enhance the fluorescent intensity response of Th6HI to duplex hybridization from 2-fold, to ˜18 fold with excitation at 425 nm, and ˜40-fold with excitation at 405 nm. Thus, the FRET pair is efficiently excited with blue laser or LEDs at 405 nm for visualization by confocal microscopy. FIG. 7 shows a normalized fluorescence excitation and emission spectra (A) of Ind6HI and Th6HI in the Narl duplex (5 μM) and the response of Th6HI (λₑₓ=425 nm) to duplex formation when hybridized with a complement strand containing Ind6HI (B). Spectra were recorded in 50 mM sodium phosphate buffer, pH 7, with 0.1 M NaCl at room temperature.

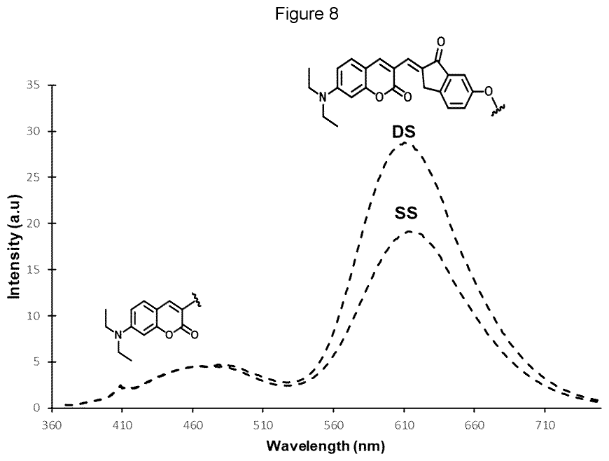

Ratiometric detection is a powerful technique often utilized in free dyes to eliminate false positives and help enhance signal to noise,²⁸ yet the incorporation of ratiometric probes into DNA is a relatively unexplored field. As such, a known fluorescent aldehyde was incorporated, which when condensed with an acceptor group can exhibit dual emission for ratiometric sensing properties (i.e., Cou). As displayed in FIG. 8, ratiometric detection of duplex formation is feasible with both Cou6HI. Upon excitation at 350 nm, dual emission can be seen at ˜ 470 nm and ˜615 nm which are respectively ascribed to the locally excited (LE) coumarin moiety and fully conjugated chalcone Upon duplex formation only fluorescence from the fully conjugated Cou6HI rotor was responsive, with little to no change in fluorescent intensity from the locally excited (LE) band. Similar results were observed by our research group when a free coumarin-hemicyanine probe displayed preferential enhancement of ICT emission over LE emission upon G-Quadruplex (GQ) DNA binding.²⁹ Reasoning for such a response from the coumarin-hemicyanine was attributed to an increase in dye planarity upon stacking interactions with the GQ structure. Likewise, the internal ratiometric probes display turn-on ICT emission upon intercalation into duplex DNA. FIG. 8 shows a fluorescence emission spectra displaying the ratiometric response of Narl (5 μM) labelled with Cou6HI to hybridization across from the prototypical abasic site (THF). Spectra recorded in 50 mM sodium phosphate buffer, pH 7, with 0.1 M NaCl at room temperature.

While the present disclosure has been described with reference to what are presently considered to be the preferred examples, it is to be understood that the application is not limited to the examples described herein. To the contrary, the present disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

### REFERENCES

(1) Haidekker, M. A.; Nipper, M.; Mustafic, A.; Lichlyter, D.; Dakanali, M.; Theodorakis, E. A. Dyes with Segmental Mobility: Molecular Rotors. In Advanced Fluorescence Reporters in Chemistry and Biology I; Demchenko, A. P., Ed.; Springer Series on Fluorescence; Springer Berlin Heidelberg: Berlin, Heidelberg, 2010; Vol. 8, pp 267-308. https://doi.org/10.1007/978-3-642-04702-2_8.
(2) Mohanty, J.; Barooah, N.; Dhamodharan, V.; Harikrishna, S.; Pradeepkumar, P. I.; Bhasikuttan, A. C. Thioflavin T as an Efficient Inducer and Selective Fluorescent Sensor for the Human Telomeric G-Quadruplex DNA. J. Am. Chem. Soc. 2013, 135 (1), 367-376. https://doi.org/10.1021/ja309588h.
(3) Nygren, J.; Svanvik, N.; Kubista, M. The Interactions between the Fluorescent Dye Thiazole Orange and DNA. Biopolymers 1998, 46 (1), 39-51. https://doi.org/10.1002/(SICI)1097-0282(199807)46:1<39::AID-BIP4>3.0.CO;2-Z.
(4) Pratihar, S.; Agrawal, R.; Pal, V. K.; Singh, A.; Govindaraju, T. Reliable Fluorometric Detection of SARS-CoV-2 by Targeting the G-Quadruplex through PH-Triggered Conformational Polymorphism. ACS Sens. 2022, 7 (2), 453-459. https://doi.org/10.1021/acssensors.1c02113.
(5) Feng, G.; Luo, X.; Lu, X.; Xie, S.; Deng, L.; Kang, W.; He, F.; Zhang, J.; Lei, C.; Lin, B.; Huang, Y.; Nie, Z.; Yao, S. Engineering of Nucleic Acids and Synthetic Cofactors as Holo Sensors for Probing Signaling Molecules in the Cellular Membrane Microenvironment. Angewandte Chemie International Edition 2019, 58 (20), 6590-6594. https://doi.org/10.1002/anie.201901320.
(6) Xu, W.; Chan, K. M.; Kool, E. T. Fluorescent Nucleobases as Tools for Studying DNA and RNA. Nature Chem 2017, 9 (11), 1043-1055. https://doi.org/10.1038/nchem.2859.
(7) Karimi, A.; Börner, R.; Mata, G.; Luedtke, N. W. A Highly Fluorescent Nucleobase Molecular Rotor. J. Am. Chem. Soc. 2020, 142 (34), 14422-14426. https://doi.org/10.1021/jacs.0c05180.
(8) Dumas, A.; Luedtke, N. W. Cation-Mediated Energy Transfer in G-Quadruplexes Revealed by an Internal Fluorescent Probe. J. Am. Chem. Soc. 2010, 132 (51), 18004-18007. https://doi.org/10.1021/ja1079578.
(9) Fang, G.; Chamiolo, J.; Kankowski, S.; Hövelmann, F.; Friedrich, D.; Löwer, A.; Meier, J. C.; Seitz, O. A Bright FIT-PNA Hybridization Probe for the Hybridization State Specific Analysis of a C→U RNA Edit via FRET in a Binary System. Chem. Sci. 2018, 9 (21), 4794-4800. https://doi.org/10.1039/C8SC00457A.
(10) Hövelmann, F.; Seitz, O. DNA Stains as Surrogate Nucleobases in Fluorogenic Hybridization Probes. Acc. Chem. Res. 2016, 49 (4), 714-723. https://doi.org/10.1021/acs.accounts.5b00546.
(11) Hövelmann, F.; Gaspar, I.; Chamiolo, J.; Kasper, M.; Steffen, J.; Ephrussi, A.; Seitz, O. LNA-Enhanced DNA FIT-Probes for Multicolour RNA Imaging. Chem. Sci. 2016, 7 (1), 128-135. https://doi.org/10.1039/C5SC03053F.
(12) Manderville, R. A.; Wetmore, S. D. C-Linked 8-Aryl Guanine Nucleobase Adducts: Biological Outcomes and Utility as Fluorescent Probes. Chem. Sci. 2016, 7 (6), 3482-3493. https://doi.org/10.1039/C6SC00053C.
(13) Mata, G.; Luedtke, N. W. Fluorescent Probe for Proton-Coupled DNA Folding Revealing Slow Exchange of i-Motif and Duplex Structures. J. Am. Chem. Soc. 2015, 137 (2), 699-707. https://doi.org/10.1021/ja508741u.
(14) Gray, M. D.; Deore, P. S.; Chung, A. J.; Van Riesen, A. J.; Manderville, R. A.; Prabhakar, P. S.; Wetmore, S. D. Lighting Up the Thrombin-Binding Aptamer G-Quadruplex with an Intemal Cyanine-Indole-Quinolinium Nucleobase Surrogate. Direct Fluorescent Intensity Readout for Thrombin Binding without Topology Switching. Bioconjugate Chem. 2020, 31 (11), 2596-2606. https://doi.org/10.1021/acs.bioconjchem.0c00530.
(15) Ebrahimi, S. B.; Samanta, D.; Cheng, H. F.; Nathan, L. I.; Mirkin, C. A. Forced Intercalation (FIT)-Aptamers. J. Am. Chem. Soc. 2019, 141 (35), 13744-13748. https://doi.org/10.1021/jacs.9b06450.
(16) Dziuba, D.; Pospíšil, P.; Matyašovský, J.; Brynda, J.; Nachtigallová, D.; Rulíšek, L.; Pohl, R.; Hof, M.; Hocek, M. Solvatochromic Fluorene-Linked Nucleoside and DNA as Color-Changing Fluorescent Probes for Sensing Interactions. Chem. Sci. 2016, 7 (9), 5775-5785. https://doi.org/10.1039/C6SC02548J.
(17) Riedl, J.; Ménová, P.; Pohl, R.; Orság, P.; Fojta, M.; Hocek, M. GFP-like Fluorophores as DNA Labels for Studying DNA-Protein Interactions. J. Org. Chem. 2012, 77 (18), 8287-8293. https://doi.org/10.1021/jo301684b.
(18) Holzhauser, C.; Wagenknecht, H.-A. DNA and RNA “Traffic Lights”: Synthetic Wavelength-Shifting Fluorescent Probes Based on Nucleic Acid Base Substitutes for Molecular Imaging. J. Org. Chem. 2013, 78 (15), 7373-7379. https://doi.org/10.1021/jo4010102.

1. A compound of the Formula (I) having the structure 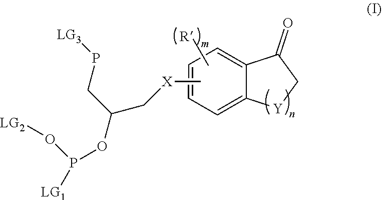
wherein
X is O or NR₁, wherein R₁ is H or (C₁-C₆)-alkyl;
Y is —C(R)(R)—, O, S, or NR₂, wherein R₂ is H or (C₁-C₆)-alkyl;
each R is independently or simultaneously H, halo, (C₁-C₆)-alkyl or (C₁-C₆)-alkoxy;
R′ is a substituent on the benzene ring and is halo, (C₁-C₆)-alkyl or (C₁-C₆)-alkoxy, and m is 0, 1, 2 or 3;
LG₁, LG₂ and LG₃ are each suitable leaving groups;
n is the integer 1, 2 or 3; and
any stereoisomers thereof.

2. The compound of Formula (I) of claim 1, wherein X is O or NH.

3. The compound of Formula (I) of claim 2, wherein X is O.

4. The compound of Formula (I) of claim 1, wherein each R is independently or simultaneously H, halo, or (C₁-C₆)-alkyl.

5. The compound of Formula (I) of claim 4, wherein each R is independently or simultaneously H, halo, or (C₁-C₃)-alkyl.

6. The compound of Formula (I) of claim 5, wherein each R is H.

7. The compound of Formula (I) of claim 1, wherein each R′ is independently or simultaneously H, halo, or (C₁-C₆)-alkyl.

8. The compound of Formula (I) of claim 7, wherein each R′ is independently or simultaneously H, halo, or (C₁-C₃)-alkyl.

9. The compound of Formula (I) of claim 8, wherein each R is H.

10. The compound of Formula (I) of claim 1, wherein LG₁ is 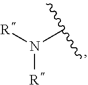
wherein
each R″ is independently or simultaneously (C₁-C₁₀)-alkyl, (C₂-C₁₀)-alkenyl or (C₂-C₁₀)-alkynyl, in which one or more carbon atoms in the alkyl, alkenyl or alkynyl groups can be replaced with an oxygen atom, or NR₁ group.

11. The compound of Formula (I) of claim 10, wherein each R″ is (C₁-C₃)-alkyl.

12. The compound of Formula (I) of claim 11, wherein LG₁ is

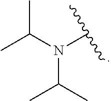 [SMILES: *N(C(C)C)C(C)C]

13. The compound of Formula (I) of claim 1, wherein LG₂ is 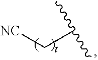
wherein
t is an integer from 1 to 6.

14. The compound of Formula (I) of claim 13, wherein LG₂ is

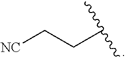 [SMILES: *CCC#N]

15. The compound of Formula (I) of claim 1, wherein LG₃ is 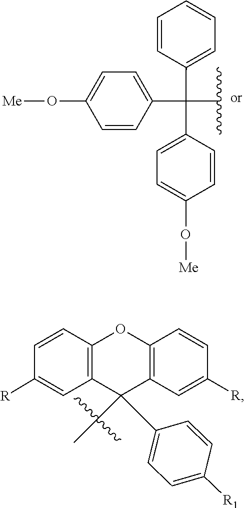
wherein
R is H or (C₁-C₁₀)-alkyl;
R₁ is H, (C₁-C₁₀)-alkyl, halo or NO₂.

16. The compound of claim 1, wherein the compound of Formula (I) is

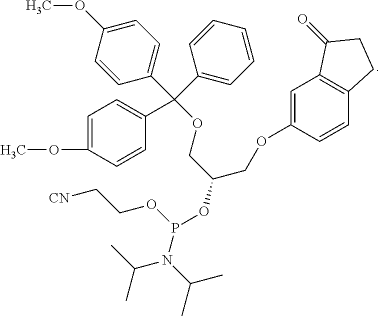

17. A compound of the Formula (II) having the structure 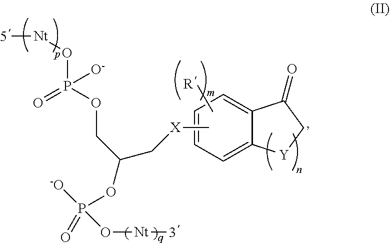
wherein,
X is O or NR₁, wherein R₁ is H or (C₁-C₆)-alkyl;
Y is —C(R)(R)—, O, S, or NR₂, wherein R₂ is H or (C₁-C₆)-alkyl;
each R is independently or simultaneously H, halo, (C₁-C₆)-alkyl or (C₁-C₆)-alkoxy;
R′ is a substituent on the benzene ring and is halo, (C₁-C₆)-alkyl or (C₁-C₆)-alkoxy, and m is 0, 1, 2 or 3;
n is the integer 1, 2 or 3;
wherein Nt is a nucleotide;
p and q are independently an integer between 0 and 1000; and
any stereoisomers thereof.

18. The compound of claim 17, wherein Nt is cytosine (C), thymine (T), uracil (U), adenine (A), or guanine (G), or derivatives thereof.

19. A compound of the Formula (III) having the structure 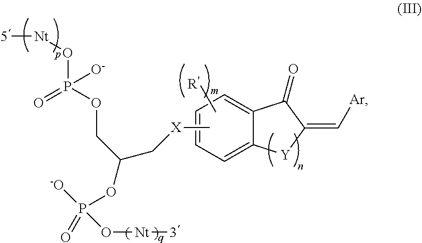
wherein,
X is O or NR₁, wherein R₁ is H or (C₁-C₆)-alkyl;
Y is —C(R)(R)—, O, S, or NR₂, wherein R₂ is H or (C₁-C₆)-alkyl;
each R is independently or simultaneously H, halo, (C₁-C₆)-alkyl or (C₁-C₆)-alkoxy;
R′ is a substituent on the benzene ring and is halo, (C₁-C₆)-alkyl or (C₁-C₆)-alkoxy, and m is 0, 1, 2 or 3;
n is the integer 1, 2 or 3;
wherein Nt is a nucleotide;
p and q are independently an integer between 1 and 1000;
Ar is an aromatic moiety; and
any stereoisomers thereof

20. The compound of claim 19, wherein the aromatic moiety is derived from

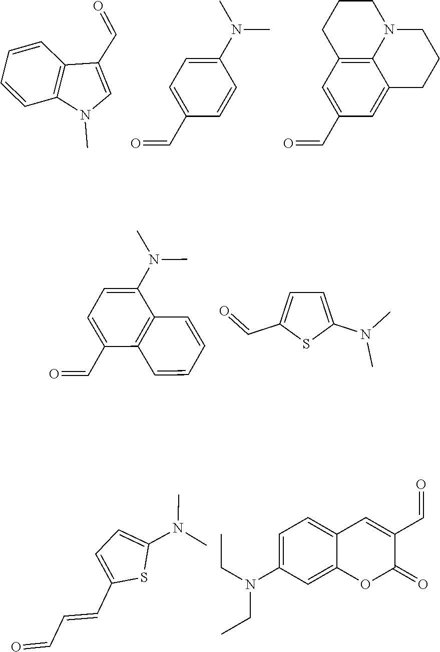

21. A method of detecting a nucleic acid, comprising
a) contacting a sample with a chalcone derivatized oligonucleotide having the structure of Formula (III) as defined in claim 19;
b) detecting a fluorescence signal at a wavelength specific for the chalcone moiety;
c) comparing the fluorescence signal of (b) with the fluorescence intensity of a control sample;
wherein detection of a signal in the sample having a fluorescence intensity greater than the control sample indicates the sample contains the nucleic acid
